(19) United States
(12) Patent Application Publication
(10) Pub. No.: US 2002/0081324 A1
(43) Pub. Date: Jun. 27, 2002

(54) Method of treating aging skin and wrinkles using a combination of growth factors that is commercially prepared or derived from one's own blood

(76) Inventor: Rebecca Wright Twine, Hempstead, NY (US)

Correspondence Address: Rebecca Wright Twine, Ph.D., M.D., 121 Cathedral Avenue, Hempstead, NY, 11550, US

(21) Appl. No.: 10/051,146

(22) Filed: Jan. 22, 2002

(51) Int. Cl. A61K 38/18; A61K 7/00

(52) U.S. Cl. 424/401; 514/2

(57) ABSTRACT

The invention is a method of treating aging skin and wrinkles by using a combination of commercially prepared growth factors, platelet derived growth factor (PDGF), epidermal growth factor (EGF), and insulin-like growth factors (IGF-I and IGF-II) incorporated into a cosmetic and/or a pharmaceutical preparation and applied to the face to stimulate skin cell renewal and fibroblasts to divide and synthesize elastin, collagen, proteoglycans, and new connective tissue, thereby reducing wrinkles, restoring elasticity, resiliency, and suppleness to the skin. The invention is also a method of treating aging skin and wrinkles using an individual's own blood to obtain the serum and plasma fractions which are rich in platelet derived growth factor (PDGF), and insulin-like growth factors, (IGF-I and IGF-II. The plasma and serum containing the growth factors are incorporated into a cosmetic and/or a pharmaceutical preparation and applied to the face to stimulate skin cell renewal and fibroblasts to divide and synthesize elastin, collagen, proteoglycans, and new connective tissue, thereby reducing wrinkles, restoring elasticity, resiliency, and suppleness to the skin.

### CROSS REFERENCE TO RELATED APPLICATIONS

N/A

### STATEMENT REGARDING FEDERALLY SPONSORED RESEARCH OR DEVELOPMENT

N/A

### REFERENCE TO A MICROFICHE APPENDIX

N/A

### REFERENCE TO SEQUENCE LISTING, A TABLE, OR A COMPUTER PROGRAM LISTING APPENDIX

N/A

### BACKGROUND

1. Field of Invention

The invention relates generally to skin, but it specifically relates to a method of treating aging skin and wrinkles by using a combination of potent mitogenic growth factors to stimulate skin cell renewal and fibroblasts to divide and synthesize new proteins that are essential to the maintenance of a healthy and youthful skin.

The first signs of aging usually show up in the skin. The face is often the first tell-tale sign of one's age. Wrinkles, the results of lost of elasticity and resiliency, occur first across the brow and around the mouth. Eventually the creases around the eyes and nose and on the cheeks become more noticeable, and the skin around the neck loosens. The skin becomes drier due to a loss of its own natural moisturizers. Thus, the cosmetic industry has inundated the market with thousands of different skin-care products that promise to reverse the track of time, especially in the face, by removing wrinkles and preventing the skin from aging. These products contain a vast array of ingredients and formulations. Yet they all claim to stop aging, and still no one stops getting wrinkles or shed a wrinkle. None of the formulations permanently alter the characteristics of aging skin or permanently restore elasticity.

The cosmetic industry constantly produces new skin-care products without any scientific basis. The cosmetic industry is not tightly regulated by the FDA. As a consquence, the industry manufactures many products without proving their claims. A cosmetic company can say a product provides all sorts of alledged benefits, from removing wrinkles to tightening pores to stmulating skin cell renewal, yet the product can contain any combination of ingredients as long as they are approved for use in cosmetics, and the benefits don't have to be proven. The only FDA restriction on cosmetics is the legal prohibition of phrases that directly state or promise a permanent change in the skin. Phamaceutical and over-the-counter drug regulations are infinitely stricter than those dealing with cosmetics.

Of all of the face skin-care products on today's market, the moisturizing creams are most popular, highly advertised, and the least expensive. The moisturizing creams simply moisturize the skin, plumping it up so lines and creases are less noticeable. Most moisturing creams contain several ingredients that encourage skin cells and layers of the skin to retain water. The ingredients include attractants (that attract water from other sources), ingredients that function as humectants and water binding agents (ingredients that retain water) barrier ingredients (which prevent water loss), and emollients (which duplicate the skin's lipid strructure). The moisturizer helps to keep the skin soft, smooth, and moist. The moisturizers do not contain ingredients that stimulate skin cell renewal and/or the synthesis of collagen and elastin by dermal fibroblastic cells. None of the moisturizing creams on today's market contain ingredients with mitogenic properties.

Face creams that contain the alpha and beta hydroxy acids are also very popular and are highly advertised. The alpha and beta hyroxy acids are classified as exfoliants. The most commonly used alpha hydroxy acids are glycolic and lactic acids because of their special ability to penetrate the skin. The most commonly used beta hydroxy acid is salicylic acid. The hydroxy acids make the skin clearer and smoother because they function to remove old dead skin cells from the surface of the skin. There have been claims by the cosmetic industry that the alpha and beta hydroxy acids stimulate the production of new skin cells. The basis for the claim is that removal of the old dead skin cells from the surface of the skin serves as a stimulus to regenerate new skin cells. The hydroxy acids are not mitogenic. They do not stimulate resting skin cells to multiply and divide. They are excellent for the purpose of maintaining good skin hygiene. A major disadvantage of the hydroxy acids is they can cause severe irritation to the skin.

There is a general consensus among cosmetic chemists and dermatologists that that major cause of aging and wrinkling of the skin is a direct result of sun exposure. According to the cosmetic chemists and dermatologist, the (UVA) rays cause wrinkles. It is reported in the cosmetic and dermatological literature that if a sunscreen product does not contain avobenzone, zinc oxide, or titaniun oxide, it cannot protect against the sun's (UVA) rays. Because of the widely held theory by cosmetic chemists and dermatologist that wrinkling of the skin is a direct result of exposure to the sun's (UVA) rays, almost all of the skin-care products on today's market contain some kind of suncreen, at least SPF 15. The sunscreens only protect the skin from (UVA) and (UVB) rays from the sun. In spite of the widely held theory among cosmetic chemists and dermatologist that the sun is the major cause of wrinkling, none of the suncreens, not even the strongest have been effective in eliminating wrinkles. The sunscreens might have a preventive role in the treament of wrinkles, but not a curative one. The chemicals that make up the sunscreens have no known mitogenic properties. They cannot stimulate skin cells to divide and multiply, and synthesize new proteins.

According to dermatologists and cosmetic chemists, (UVA) rays from the sun cause the build-up of free radicals in the deep layers of the skin which, in turn, cause damage to the skin's support structures, notably collagen.and elastin. Thus, the skin looses elasticity and resiliency. It is well established in the biochemistry literature that antioxidants slow down free radical damage. Because of this concept, the incorporation of antioxidants into skin-care products have been exploited by the cosmetic industry. Thus, the market has become proliferated with skin-care products enriched with antioxidants. Almost all moisturizers contain a combination of antioxidants. Face creams containing antioxidants are popular and receive much attention in fashion magazines and in infomercials. Fashion magazines have heralded the elimination of free-radical damage as the fountain of youth for the '90s. The most common antioxidants used in skin-care products are vitamins A, C, and E. Less commonly used antioxidants in skin-care products are, superoxide dismutase, flavenoids, beta carotene, selenium, and zinc. According to experts in the field of antioxidant research, there is no conclusive scientific evidence that antioxidants really prevent or erase wrinkles. Nevertheless, cosmetic companies still load the market with skin-care products containing almost all available antioxidants. None of the antioxidants have mitogenic properties They cannot stimulate resting skin and fibroblstic cells to divide, multiply, and synthesize new proteins. Moisturizers enriched with antioxidants may be helpful in improving the appearance of one's skin, but they do not prevent or eliminate wrinkles.

Several face creams containing estrogenic substances are marketed with the claim that they stimulate skin cell renewal. Most of the creams containing estrogenic substance are extracts from soy bean plants (tofu) and wild yams. The FDA has control over the amount of commercially prepared estrogen that cosmetic chemists are permitted to incorporate into a skin-care product. The amount is relatively small and, if added to a skin care product, the benefit is little. Cosmetic creams containing small amounts of estrogen and other hormones cause the skin to swell slightly, which makes fine lines less noticeable for a short time after treatment. Estrogen is classified as a female hormone. Estrogen is not considered a mitogen, in spite of the fact, it promotes the growth of certain cancers. According to the FDA, estrogen is not a remedy for the treatment of wrinkles.

Numerous skin formulations that contain DNA and RNA have also proliferated the market. The manufacturers of such creams claim that they can “rejuvenate” cells. Their theory is that DNA and RNA can stimulate skin cells that have stopped reproducing in aging skin to divide and synthesize new proteins, especially collagen. Supposedly, DNA initiates the cell cycle and RNA initiates the synthesis of skin proteins. The theory is much more impressive than the effect is. By adding RNA to a cream and applying it to the face does not stimulate skin cells to synthesize new proteins. DNA and RNA are classified as nucleic acids. DNA and RNA are not mitogenic substances. Skin creams that contain DNA and RNA are not effective in the treatment of aging skin and wrinkles.

The cosmetic industry has inundated the market with face creams containing collagen. Collagen is the structural protein that gives skin its resilency and support. Scientific-sounding ads are eveywhere in the cosmetic market place, in fashion magazines, and in infomercials. These ads are deceptive. Since the protein collagen is a major component of skin cells, it sounds logical that adding it to a product would help restore the skin. The real truth is that collagen molecules are too large to pass through the epidermis to reach the deeper layers and be absorbed into the skin. Creams containing collagen act as moisturizers, in that they sit on the surface of the skin combating dryness. In addition, the cosmetic industry has marketed face creams that contain a combination of elastin and collagen. These creams are manufactured under the idea, such as “replace what the skin is deficient or as lost” due to aging. The manufacturers claim that the creams make the skin firmer and more elastic. The truth is that collagen an elastin molecules are too large to penetrate the skin. These creams are not effective in the treatment of wrinkles. They act as moisturizers by combating dryness, like other, less expesive moisturizers.

Retinoic acid is the only face cream that seems to work effectively in combating wrinkles and improving the appearance of aging skin. Retinoic acid is a derivative of vitamin A. It is available by prescription as Retin-A. According to the dermatological literature, Retin-A thickens the epidermis, which tends to become thin with age; speed up exfoliation; stimulates blood circulation and produce new blood vessels, giving the skin its pinker tone; and stimulates collagen production in the dermis layer, which helps firm it up. However, Retin-A has to be used indefinitely. It usually takes 3 to 4 months to see any significant changes. The major drawbacks using Retinoic acid in the treatment of wrinkles are, redness and irritation of the skin, scaly patches and itching of the skin, and inresed sensivity of the skin to sun damage. Some people cannot tolerate the irritation to the skin and simply stop using it before they reap the benefits. Patients who undergo treatment with Retin-A for wrinkles or acne must use large amounts of sunscreens. The irritation to the skin caused by Retinoic acid is probably due to its strong acidic properties. Retinoic acid is the carboxylate form of vitamin A₁¹. There are many face creams on the market that contain vitamin A and retinol. They do not have the same effect as retinoic acid.

Collagen injections have been approved by the FDA for the treatment of wrinkles The collagen injections are administered to patients by dermatologist. Purified injections of bovine collagen are injected into major creases or wrinkles on one's face. Collagen injections are effective in smoothing out wrinkles and skin creases. The major disadvantage with collagen injections are that they can cause redness and swelling, and in some instance severe allergic rections. Other risks with collagen injections are contour irregularities, infection, or local abcesses. Collagen injections are contraindicated in patients with immunological disorders. The effects usually lasts for four month to a year because the body absorbs the collagen.

Another injectable substance used to treat wrinkles is fibrel. Fibrel is prepared from a gelatin extracted from the patient's own plasma and, combined with gelatin and the clotting agent aminocaproic acid that stimulates the natural production of collagen at the site of the injection. The material is injected until the wrinkle is elevated; the injections are given one or two weeks apart. The major drawbacks of fibrel are that it causes contour irregularties, allergic reactions, swelling and redness, burning and bruising at the site of injection. It usually takes three months for new collagen to form at the site of injection. As with collagen, overcorrection is necessary because there is some absorption by the body.

One of the newest techniques used to treat wrinkles is botox (botulinum toxin), a purified form of toxin that causes botulism. Injected into the face, the substance temporarily and partially paralyzes the muscles underneath frown lines, giving the face a smoother, less furrowed look. The treatment is claimed to be effective. Experts report up to a 90% reduction in wrinkles The major disadvantges of the treatment are that it is costly and gives the individual a lifeless look. The cost is approximately $500 to $1,000 per treatment.

The invention is a method of treating aging skin and wrinkles with growth factors. The invention is based on a sound scientific rationale. As the skin ages, the cells in the deeper layers of the skin become inactive, and enter into the resting phase (G0) of the cell-division cycle. The skin cells can remain in the resting (G0) phase of the cell cycle throughout the life of an individual. The rationale for using growth factors to treat aging skin and wrinkles is because they are potent mitogens, and can stimulate resting skin and fibroblastic cells to migrate from G0 to the G1 phase of the cell cycle, where the process of cell regeneration has its beginning ^{2,3}. In addition, certain growth factors specifically target skin and fibroblastic cells making up the epidermis and dermis, respectively. The skin and fibroblastic cells have high-affinity receptors located on their surfaces for the growth factors that control their growth, division, and reproduction. The growth factors stimulate the activation of the genes in their target cells to differentiate, grow, divide, and to synthesize an abundant array of skin proteins. Thus, restoring elasticity, resiliency, and suppleness to aging skin and reducing wrinkles. In all probality, the skin and fibroblastic cells in the deeper layers of the skin enter into a resting stage because there is an age associated decline in the production of the growth factors that promote their growth, differentiation, and reproduction.

It was noted earlier that most of the face creams in today's market place are ineffective in the treatment of wrinkles, except for retinoic acid (Retin-A.). The mode of action of growth factors in the treatment of aging skin and wrinkles are similar to that of retinoic acid. However, retinoic acid is not classified as growth factor. Retinoic acid and growth factors both regulate gene expression, and the proteins produced from genes activated by retinoic acid and growth factors stimulate cellular growth, differentiation, and reproduction. However, they diverge in the manner in which they activate the genes. Retinoic acid behaves like a steroid hormone¹. It asssociates with receptors in the nucleus of target cells, and the receptor-retinoic acid complex binds to response elements in DNA, stimulating the transcription of genes¹. The receptor for retinoic acid belong to the steroid-thyroid receptor superfamily¹. On the other hand, growth factors do not enter the cells¹. Growth factors react with receptors on the cell surface, stimulating reactions that generate second messengers inside the cell that ultimately activate genes¹. It appears that retinoic acid has intracellular receptors in skin cells. The growth factors should have a similar effect on skin cells of the epidermis and fibroblastic cells in the dermis layer as retinoic acid. It was noted earlier that growth factors have receptors located on skin fibroblastic cells. However, the growth factors may have a greater impact in the treatment of aging skin and wrinkles than retinoic acid because they are potent mitogens. The impact could be even much greater than retinoic acid because a combination of different growth factors can be employed, all of which target skin and fibroblastic cells, and cooperate synergistically to induce them to differentiate, multiply, and synthesize new proteins.

A major advantage of using growth factors to treat aging skin and wrinkles is that they are small polypeptides and would unlikely cause skin irritation as seen with retinoic acid; there would also be increased compliance by individuals who are undergoing treatment for aging skin and wrinkles because they would not fear the skin reactions seen with retinoic acid.

Another advantage of using growth factors to treat aging skin and wrinkles is that individuals would not have to use large amounts of sunscreens because of an acquired heightened sensitivity to sunlight.

Still another advantage of using growth factors to treat aging skin and wrinkles is that a combination of them can be incorporated into a cosmetic and/or a pharmaceutical preparation, yielding a faster cosmetic result.

A further advantage of using growth factors to treat aging skin and wrinkles is that they are natural to the body and play physiological roles in the maintenance of a healthy and youthful skin; they a re specific for the cells they target and would not interfere with other cellular functions in the body.

A more further advantage of using growth factors in the treatment of aging skin and wrinkles is that they are small molecules and can readily penetrate the deep layers of the skin.

A major advantage of using one's own blood to treat aging skin and wrinkles is because blood contains a combination of growth factors that can be incorporated into a custom skin cream formulation consisting of an abundance of amino acids, the basic building blocks required for the synthesis of skin proteins, e.g., the individual would ingest a protein-rich meal just before his or her blood sample is drawn; active skin and fibroblastic cells that have been stimulated to divide by the growth factors will require large amounts of amino acids; amino acids are very small molecules and can be delivered simultaneously with the growth factors to the deeper layers of the skin.

Another advantage of using one'own blood and incorporating it into a skin care formulation is because it contains nutrients, e.g. .the essential fatty acids, proteins, vitamins, antibodies, etc.; the formulation will not only contain the growth factors, but would be rich in nutrients.

### BRIEF SUMMARY OF THE INVENTION

The invention is a method of treating aging skin and wrinkles by using a combination of potent mitogenic growth factors (platelet derived growth factor (PDGF), epidermal growth factor (EGF), and insulin-like growth factors (IGF-I and IGF-II) that are incorporated into a cosmetic and/or a pharmaceutical preparation to stimulate resting target skin cells in the basal layer of the epidermis and fibroblastic cells in the dermis to divide and synthesize new proteins (keratin, elastin, collagen and proteoglycans) and to form new connective tissue. The growth factors promote the transition of resting (G0) skin and fibroblastic cells to enter into the G1 phase of the cell-division cycle ². A combination of growth factors are used to overcome G1 restriction points in the cell cycle ^{2,3}.

The invention consist of two different methodologies for treating aging skin and wrinkles: (1) An autologous method—the use of one's own blood (plasma and serum) containing the growth factors PDGF, IGF-I, and IGF-II, the mixture of which is then incorporated into a cosmetic or a pharmaceutical preparation and applied to the face according to directions and (2) the use of a combination of commercially prepared growth factors (PDGF, EGF, IGF-I and IGF-II) all of which are incorporated into a cosmetic and/or a pharmaceutical preparation and applied to the face according to directions.

The rationale for using an individual's own blood to treat aging skin and wrinkles is because it is rich in a combination of growth factors, PDGF, IGF I, and IGF II ^{2,4,5}. These growth factors are potent mitogens that target fibroblasts, cells of mesodermal orgin, and epithelia². All three of the cells play very important and specific roles in the maintenance of a healthy and youthful skin.

### DETAILED DESCRIPTION OF THE INVENTION

The Skin:

The skin is made up of two layers, the epidermis and the dermis. The epidermis is a thin, relatively transparent layer which itself can be divided into three layers-the basal on the bottom (just next to the dermis), the squamous cells, and on the outer surface (stratum corneum) which consists of dead skin cells made of a protein called keratin. The outer cells are regularly sloughed off and replaced by newer skin cells, which have been manufactured in the blood enriched basal layer of the epidermis. The new skin cells rise to the outer surface in the course of a month or so, gradually dying as they become increasingly thinner and flatter. In this way the skin is a naturally replenishing organ. The stratum corneum makes the skin tough and water proof, and its function is so important that 95% of the skin cells are involved in producing it. The stratum corneum constantly sheds cells, between two and three billion cells or cellular fragments daily. A small percentage of the cells in the epidermis produce melanin, the dark pigment that is responsible for skin color and that helps to protect the skin from ultraviolet light.

The function of the cells in the basal layer of the epidermis is to continually renew the life of one's skin. They divide, and while the newly created cells are carrying on life processes the original cells are carried progressively to the surface of the skin. Because they cannot survive exposure to the air and water, they grow old and eventually die. Then they are gradually shed. It is because of this very efficient system of sloughing off the old, dead skin only to reveal new, fresh skin that one is able to maintain a soft, youthful-looking skin.

There is an increased accumulation of dead cells on the surface of aging skin. The skin cells are less frequently renewed and shedded in contrast to the skin of younger individuals. As the skin ages, the cells become less active and enter into a resting phase of cell division. The dead skin cells accumulate on the surface of the skin and gives it a sand paper to a leathery appearance. Aging skin becomes discolored because of the build up of dead skin cells laden with pigment on its surface. Thus, growth factors that target epithelial cells and stimulate them to divide and multiply could restart the renewal process, the production of new skin cells, and sloughing off of the old dead skin cells. As a result, the surface of aging skin can be rejuvenated or markedly improved.

The dermis lies just beneath the epidermis. It is rich in glands and blood vessels. The dermis is where the glands do their work. The sebaceous glands, or oil glands, are in charge of manufacturing sebum, one of the body's own moisturizing factors. The purpose of the sebum is to prevent evaporation of the water in the skin, and also to protect the body from absorbing excessive moisture. As one ages, the skin tends to become drier and less elastic. This occurs in part because of a decline in the production of oil and sweat by the glands, as well as a general decline in the whole skin renewal process. Dry skin is more prone to to wrinkling than oily skin. The dermis is also where the sweat glands produce perspiration, which seeps through the pores to help maintain a constant temperature in the body, and to help keep the skin moisturized.

The dermis is composed primarily of three kinds of proteins—collagen, elastin, and reticulin^{6,7}. These proteins are responsible for the support and elasticity of the skin^{6,7}. It is collagen and elastin that enables one's skin to snap back into shape after being stretched or pulled. Collagen and elastin together give skin its strength, as well as its supple, stretchy properties. The reticulin fibers hold together bundles of collagen fibers^{6,7}.

The dermis also contains specialized cell called fibroblasts. The fibroblasts are of mesodermal orgin. The fibroblasts synthesize collagen and elastin³. The collagen and elastin are secreted extracellularly by fibroblasts into the intercelluar matrix, the connective tissue component of the dermis^{3,6,7}. Elastin and collagen are the primary substances in the connective tissue^{3,6,7}. Collagen is a fibrous protein that has very high tensile strength. It is the major fibrous component of skin^{3,6,7}. The basic structural unit of collagen is tropocollagen, which consist of two α 1 chains and and an α 2 chain, each about 1000 residues long^{6,7}. Collagen is rich in hydroxyproline and hydroxylysine, which occur in very few other proteins. The amino acid sequence of collagen is highly distinctive: nearly every third residue is glycine^{6,7}. Elastin is an insoluble, rubber-like protein in elastic fibers of connective tissue of skin^{6,7}. Elastin like collagen is rich in proline and glycine^{6,7}. In contrast, to collagen, elastin contains very little hydroxyproline and no hydroxylysine^{6,7}.

Collagen gives connective tissue strength^{6,7}. Elastin gives skin its elastic properties, allowing the skin to stretch and spring back into place. Elastin becomes less elastic with age, the skin becomes looser. Collagen and elastin is very important to the youthful look of one's skin.

Wrinkles are defined as a crease or furrow in the skin. Wrinkles are caused by decreased production of collagen and elastin by fibroblasts or by overexposure of the skin to ultraviolet rays from the sun. As the natural process of aging takes place, there is a decreased production of collagen and elastin by fibroblasts. As a result, the dermis becomes thinner, which in turn, leads to the develoment of furrows, wrinkles, and easy brusibility of the skin. Much of the aging of the face includes not only wrinkles but also prolapse downward of the subcutaneous tissue. In addition, the layer of subcutaneous fat tends to disappear, and the skin becomes transparent, with blood vessels appearing more prominently. In addition, small liver spots (areas of darker pigmentation) may develop on the face as well as the hands. The hyperpigmented spots are more visible in light-skinned individuals. As the skin ages, the fibroblastic cells of the dermis become inactive, almost to a complete “shut down. The cells stop dividing and an enter into a resting phase called the GO phase of the cell cycle. Cells may stay in this phase indefinitely. Thus, any cosmetic and/or pharmaceutical agent aimed at stimulating fibroblast cells to divide and multiply with a concomitant increase in the synthesis of collagen and elastin fibers could have a significant beneficial effect on aging skin. Thus, restoring it to a healthy and youthful state.

The cell renewal process affects the appearance of the skin and does not contribute to the formation of wrinkles. Cell regeneration and migration from the lower layers to the surface has nothing to do with the way skin wrinkles. The cell renewal process does not affect the collagen and elastin fibers that support the skin nor does it affect the intercellular structure between skin cells. The production of collagen and elastin by specialized cells in the dermis is not related to the regeneration and movement of skin cells. The substance that exist between the skin cells are affected by aging and possibly by sun damage.

The extracellular space in animal tissues is filled with a gel-like material, the intercelluar or extracellular matrix, also called ground substance^{3,6,7}. The extracellular matrix holds the cells of a tissue together and provides a porous pathway for the diffusion of nutrients and oxygen to individual cells^{3,6,7}. The ground substance is best defined as an intercellular matrix from which all the formed elements of connective tissue, for example, fibroblasts are believed to originate³. The ground substance is a continous nonfibrillar matrix³. The intercellular matrix is composed of an interlocking meshwork of heteropolysaccharides and fibrous proteins^{3,6,7}. The heteropolysaccarides are called glycoaminoglycans^{6,7}. The glycoaminoglycans are a family of repeating dissacharide units^{6,7}. One of the two monosaccarides is always either N-acetylglucosamine or N-acetylgalactosamine^{6,7}. The other unit in most cases is a uronic acid, usually glucoronic acid^{6,7}. In some glycoaminoglycans, one or more of the hydroxyl groups of the sugar is esterfied with sulfate^{6,7}. The combination of these sulfate groups and the carboxylate groups of the uronic acid residues gives the aminoglycans a very high density of negative charges. To minimize the the repulsive forces among neighboring charged groups, these molecules assume an extended conformation in solution^{6,7}. As a result, these long,thin molecules form high viscosity solutions^{6,7}. The glycoaminoglycans are attached to extracellular proteins to form proteoglycans^{6,7}. The proteoglycans form enormous aggregates in which polysaccharide makes up most of the mass, often 95% or more^{6,7}. Interewoven with these enormous extracellular proteoglycans are the fibrous proteins collagen and elastin, which form a cross-linked meshwork that gives the whole extracellular matrix strength and resilency^{6,7}. After the proteoglycans are synthesized, they are secreted into the extracellular matrix.^{6,7} They have hydrating and lubricating properties because they carry very high density of negative charges^{6,7}. It appears that proteoglycans play a significant role in keeping the skin naturally lubricated and moisturized. As the skin ages, it becomes drier. It was noted earlier that dry skin is more prone to wrinkling than oily skin. As the skin ages, there is decreased production of the proteoglycans by cells in the intercellular matrix. As a result, the skin looses its natural moisturizing and lubricating capability. It was also noted earlier that there is decreased production of sebum by aged skin. The sebum prevents the evaporation of water from the skin. The decline in the production of sebum together with decreased synthesis of proteoglycans in aging skin causes it to become less supple. Thus, any cosmetic or pharmaceutical agent aimed at inducing cells in the intercellular matrix to increase the synthesis of proteoglycans could significantly improve the appearance of aging skin, and possibly restore it to a youthful state.

Growth Factors:

Proto-oncogenes control normal growth and cell division¹. These gene encode growth factors, growth factor receptors, transcription receptors, or other proteins involved in promoting cell growth. Normally cell division is highly regulated by a family of growth factors, proteins that cause resting cells to undergo division, and in some instances differentiation¹. Some growth factors are cell-type specific, stimulating division of only those cells with specific receptors. While other growth factors are more general in their effects.

The growth of human cells involve DNA replication and cell division. Growth factors are polypeptides that regulate gene expression, but do not enter into cells¹. The growth factors bind to receptors on the cell surface, triggering events within the cell that stimulate growth and division. The growth factor receptors are glycoproteins, with an external, transmembrane, and a cytoplasmic domain^{1,2,5}. The binding of a factor to the external domain of a receptor has two effects: It changes the cytoplasmic domain, generating an internal signal that alter cellular chareteristics, and it initiates a series of events that lead to the disappearance of the receptor (down regulation)^{2,5}. The binding of the factor to the external domain activates a protein kinase with specificity for tyrosine in the cytoplasmic domain^{2,5}. Such receptors include epidermal growth factor (EGF), platelet derived growth factor (PDGF), and insulin-like growth factor (IGF-I)^{2,5}. The receptor itself can act as a protein kinase, phosphorylating tyrosine residues of intracellular proteins².

Growth factors are potent mitogens^{2,4}. Growth factors stimulate cells to divide and multiply by overcoming the blocks to replication that occurs during the G0-G1 phase of the cell cycle². The cell cycle is divided into four phases: G1, which precedes DNA replication; S, during which DNA synthesis occurs; G2 and M, when mitosis and finally cell division occur^{1,2,3}. The distribution of the cells of a culture in the various phases of the cell cycle can be ascertained by the tecnique of cytofluorometry². From cells with a doubling time of 18 hours, typical lengths of the various periods are G1, 10 hours; S, 6 to 7 hours; G2, 1 hour; and M about ½ hour^{2,} Progress from one phase to another results from accumulation of specific substances within cells².

The G1 period, which can vary enormously in length, depending on the cell type and the growth conditions within cells, causing the G1 lengths of different cells to follow a “single event” distribution². There is growth restriction in the G1 phase². The great variation in length of the G1 phase accounts for most of the variation in the total cycle time. During the G1 phase, the growth of untransformed cells must proceed through restriction points that are overcome by certain growth factors in the cell's medium². The single growth factor may be sufficient to overcome growth restriction in the G1 phase of the cell cycle, such as PDGF which promotes the transition of fibroblastic cells from G0 to G1, but additional growth factors such as IGF factors are needed to overcome restriction points in the G1 phase.²

It was demonstrated by the technique of Cytofluorometry that in quiescent untransformed cultures the cells are arrested in the G0 phase². It was further shown by cytofluorometry that cultures manifested a reduced uptake of glucose, phosphate, and a reduced synthesis of RNA and proteins². It was also shown by cytofluorometry that the cells displayed faster protein degradation and that most of the messenger RNA was free, rather than in polysomes². Thus, suggesting a decrease in the synthesis of proteins by resting (G0) cells.

The natural aging of skin appears to be caused by decreased activity of the skin cells and fibroblasts. With aging, a large percentage of the skin cells and fibroblasts remain in a quiescent state, the G0 phase of the cell cycle. As a result, there is a marked decrease in the synthesis of proteins e.g., elastin, collagen, and proteoglycans,etc. As a consequence, the skin loses its moisture, elasticity, and resilency which ultimately causes sagging and wrinkles. In addition, the appearance of the skin is adversely affected by a decline in the regeneration and migration of skin cells (cell renewal) from the basal layer of the epidermis to the surface. Thus, the skin becomes rougher and with patchy areas of hyperpigmentation.

Human serum and plasma are rich in growth factors that target epithelial, mesodermal, and fibroblast cells². The serum is rich in platelet derived growth factor (PDGF) and the plasma have high concentrations of insulin-like growth factors (IGF I and IGF II). These growth factors have the capability of binding to receptors on the surface of skin and fibroblast cells and stimulate them to divide and multipy, thereby inducing them to synthesize proteins, e.g., keratin, collagen and elastin, respectively. These growth factors can stimulate quiescent populations of skin cells and fibroblasts in aging skin to migrate from the G0 phase to the G1 phase of the cell cycle. A combination of growth factors can cooperate collectively in overcoming the subsequent G1 restriction points.

Platelet Derived Growth Factor (PDGF): Normal fibroblast division is regulated by by the interaction of several oncogenes which code for normal growth factors and their receptors¹. Important for fibroblast cells is the growth factor PDGF^{2,3,4}. PDGF is a small glycoprotein made up of two chains and is stored in the alpha granules of platelets and is released during blood clotting^{2,3}. The serum contains more PDGF than the plasma². The PDGF has intrinsic tyrosine kinase activity when activated by an appropiate ligand^{2,5}. The binding of PDGF to high-affinity receptors on the surface of fibroblast cells signal those in the G0 phase to enter the G1 phase of the cell division cycle (G1,-S-G2-M-G1)^{2,3}. According to the literature, PDGF requires the cooperation other growth factors, for example, the epidermal growth factor (EGF) and insuln-like growth factors (IGF-I and IGF-II) in overecoming the G1 restriction points^{2,3}. Thus any cosmetic and/or pharmaceutical preparation utilizing PDGF to treat aging skin and wrinkles should include IGFs and EGF. The IGFs and EGF also target fibroblasts.

Insulin-Like Growth Factors (IGF-I and IGF-II):

The insulin-like growth factors are a family of polypeptides that stimulate proteoglycan synthesis in cartilage and DNA synthesis and cell replication in a variety of cell types and that demonstrate insulin-like activity³. Human plasma contains large amounts of insulin-like activity that does not reside in insulin itself, so called non-supressible insulin-like activity, NSILA^{1,3,5}. It is divisible into chemically and biologically related polypeptides, NSILA-I and NSILA-II^{3,5}. The insulin-like growth factors are also known as the somatomedins. The insulin-like growth factors share structural homologies with proinsulin and both have substantial insulin-like growth activity^{1,3,5}. Thus, they are designated, insulin-like growth factor-I (human IGF-I or somatomedin-C ) and (insulin-like growth factor-II (human IGF-II), or somatomedin A)^{1,3,5}.

IGF I is a single-chain basic peptide having 70 amino acids. These two peptides are identical to insulin in half of their residues. In addition, they contain a structural domain that is homologous to the C-peptide of proinsulin^{1,3,5}. A broad spectrum of normal cells respond to high doses of insulin by increasing thymidine uptake and initiating cell propagation. In most instances, IGF-I causes the same response as insulin in these cells but at signficantly smaller, and more physiological concentrations^{3,5}. Thus, the IGFs are more potent than insulin in their growth promoting actions. Unlike most hormones, the somatomedins are synthesized in many tissues and act via ubiquitous (type I and II) receptors¹. The liver is the major source of of circulating IGF-I, but other tissues contribute to circulating somatomedins ¹. In contrast to insulin the IGFs circulate in the plasma in complex with a family of binding proteins that extend the serum half-life of the IGF peptides, transport the IGFs to target cells, and modulate the interaction of the IGFs with surface membrane receptors¹. According to the literature, the carrier proteins are essential for somatomedin actions by facilitating delivery to the receptors¹.

According to the literature, the IGFs are active via autocrine and paracrine as well as endocrine routes¹. IGF-I stimulates cell propagation and growth by binding to specific IGF-I receptors on the plasma membrane of target cells, rather than binding to growth hormone receptors¹. Like insulin, the intracellular portion of the plasma membrane receptor for IGF-I (but not for IGF-II) has intrinsic tyrosine kinase activity¹.

It is reported in the literature that tyrosine phosphorylation initiates the process of cellular replication and growth¹. This was based on the fact that the receptors for insulin and a number of other growth factors have intrinsic tyrosine kinase activity. Most of the cells of the body has mRNA for IGF, but the liver has the greatest concentration of these messengers followed by the kidney and heart¹. The synthesis of IGF-I is regulated for the most part by growth hormone (GH)¹, whereas hepatic production of IGF-II is independent of GH levels in the blood¹. The growth hormone binds to cell surface receptors and stimulate IGF-I production and release from the liver and other tissues¹. The IGF-I then binds to cell surface receptors and stimulate the phosporylation of proteins that cause mitosis and growth¹.

IGF-I and IGF-II mRNA are localized in mesenchymal and fibroblast cells in the interstitial connective tissue⁵. Fibroblasts and skin cells synthesize both IGFs⁵. As the skin ages, there is decreased synthesis of the growth factors by fibroblasts and skin cells. Thus, there is a decline in skin cell renewal and the synthesis of collagen and elastin by the fibroblasts. As the skin ages, the skin cells and fibroblasts become quiescent and and enter the G0 phase of the cell division cycle. A cosmetic and/or a pharmaceutical preparation containing a combination of growth factors, e.g., PDGF and IGFs could enhance the synthesis of collagen and elastin by fibroblasts, and thereby “jump start” the entire skin renewal process.

According to the literature, the IGFs are not just potent mitogens, but they also stimulate the synthesis of proteoglycans³. The proteoglycans are the large hydrating and lubricating protein aggregates that make-up the intercellular matrix^{6,7}. The IGFs stimulate [³⁵S] sulfate incorporation into cartilage, and because of this they are also called the sulfation factor^{1,3}. The proteoglycans carry a very high density of negative charges due to the sulfation of the glycoaminoglycans (repeating units of disaccharides) that are attached to them and other polar groups^{6,7}. The charge groups and other polar groups in the sugar derivatives can bind water molecules. The combination of open structure due to water repulsion and strongly hydrophilic result in a molecule with a very large domain, Thus, it occupies a relatively large volume of solution compared to a molecule of equal mass with a compact structure. The high density of negative charges on the proteoglycans account for their hydrating and lubricating properties^{6,7}. Proteoglycans are the skin's natural moisturizers and lubricants^{6,7}. Any cosmetic or pharmaceutical preparation that contains IGFs could stimulate the synthesis of proteoglycans, which are then secreted into the extracellular matrix of aging skin. The elastin and collagen fibers as well as the intercellular matrix become more hydrated. As a result, wrinkles would become less pronounced, and the skin more supple.

According to the literature, after age 20-30, serum IGF-I levels demonstrate a gradual-and progressive age-associated decline⁵. It was stated in the literature that by age 1, adult levels of IGF-II are attained, with little if any subsequent decline, even up to the seventh or eighth decade of life. It was noted earlier that the synthesis of IGF-I is largely controlled by growth hormone (GH)^{1,5}. It is well established in the literature that as one ages, there is a decline in the blood levels of growth hormone⁵. Thus, the serum levels of GH and IGF-I decline in a parallel fashion⁵. IGF-I may be more associated with aging of the skin than IGF-II. since skin ages in the presence of relatively high serum levels of IGF-II. Thus any cosmetic or pharmaceutical preparation aimed at treating aging skin and wrinkles should include IGF-I.

Epidermal Growth Factor (EGF):

EGF is a potent mitogen. EGF is a single polypetide chain of 53 amino acid residues that contain three intramolecular disulfide bonds⁵. EGF is synthesized in the submaxillary gland^{2,5}. It is found in very high concentrations in the urine^{3,5}. EGF targets fibroblasts and epithelia³. The target cells have high-affinity receptors for EGF on their surfaces². The receptors have intrinsic tyrosine kinase activity^{2,5}. EGF promotes the transition of epithelia and fibroblasts that are in the resting stage (G0 phase) to enter into the G1 phase of the cell-division cycle^{2,3}. According to the literature, EGF enhances a number of events that are part of the mitogenic response during wound healing³. It was stated in the literature that a local application of EGF enhanced the accumulation of cells and ground substance in experimental wounds, but did not heal it⁸. It appears that EGF may have a significant beneficial effect in the treatment of aging skin and wrinkles by promoting skin cell renewal and production of the intercellular matrix (ground substance).

Principal Mode of Action of Growth Factors in the Treatment of Aging Skin and Wrinkles:

The growth factors, PDGF, IGF-I, IGF-II, and EGF are potent mitogens, at nanogram and picogram levels can induce target cells to multiply, form new connective tissue, and synthesize new proteins. They have high-affinity receptors on the surface of target cells, and have intrinsic tyrosine kinase activity, except for IGF-II.

The platelet derived growth factor (PDGF) target fibroblasts and cells of mesodermal orgin². PDGF promotes the transition of fibroblasts from G0 to G1². Other growth factors, e.g., such as insulin-like growth factors and EGF, can cooperate with PDGF in overcoming subsequent G1 restriction point². After fibroblastic cells enter into the cell division cycle, they begin to synthesize new elastin and collagen fibers and secrete them into the extracelluar matrix. An increased synthesis of elastin and collagen in aging skin could repair and reduce wrinkles.

Principal Mode of Action of Insulin-Like Growth Factors (IGF-I and IGF-II) in the Treatment of Aging Skin and Wrinkles:

The IGFs target fibroblasts and epithelia. The IGFs promote the transition of skin fibroblastic cells from G0 to G1². The IGFs can also cooperate with PDGF in overcoming the subsequent G1 restriction points². In addition, the IGFs stimulate skin cell renewal, the regeneration of skin cells in the basal layer of the epidermis and their migration to the surface. The stimulation of skin cell renewal in aging skin can result in a smoother and a clearer skin. IGFS induce resting fibroblastic cells in aging skin to synthesis new elastin and collagen, thereby resulting in a more firmer, elastic, and resilient skin and a reduction in wrinkling.

The IGFs also stimulate the synthesis of proteoglycans^{1,3}. The proteoglycans are the skin's natural lubricants and moisturizers^{6,7}. The IGFs stimulate the incorporation of sulfate groups on sugar residues of the glycoaminoglycans that are attached to proteoglycans^{1,3}. The negative charges from the sulfate and carboxylate groups of the sugar residues give proteoglycans their powerful hydrating and lubricating properties^{6,7}. An increase in the synthesis of proteoglycans in aging skin would cause it to become more supple, and with less wrinkling. Thus, a cosmetiic and/or a pharmaceutical preparation containingg IGFs would be beneficial in the treatment of aging skin and wrinkles.

Principal Mode of Action of Epidermal Growth Factor (EGF) in the Treatment of Aging Skin and Wrinkles:

EGF is a potent mitogen. EGF is a single polypeptide chain of 53 amino acid residues that contain thre intramolecular disulfide bonds. EGF is synthesized in the submaxillary gland. EGF is not found in high concentrations in blood. It is in very high concentrations in the urine. EGF target fibroblasts and epithelia. The target cells have high-affinity receptors on their surface for EGF. The EGF receptors have intrinsic tyrosine kinase activity^{2,5}.

EGF promotes the transition of resting fibroblasts from G0 to G1 in aging skin, thereby stimulating them to multiply and synthesize new collagen and elastin. In addition, EGF can cooperate with PDGF in overcoming G1 restriction points in the cell cycle., thereby enhancing the growth of fibroblastic cells². Any cosmetic and/or a pharmaceutical preparation containing EGF in combination with PDGF and IGFs could have a “rejuvenating” effect on aging skin. Thus, making it more supple, elastic, resilient, firmer, clearer, and smoother.

Ideally, a combination of growth factors should be included in any cosmetic and/or a pharmaceutical preparation aimed at treating aging skin and wrinkles. The rationale for using a combination of growth factors is that they cooperate collectively in overcoming the G1 restriction points in the cell cycle, thereby reducing the length of time that G0 cell recruits spend in the G1 phase. Thus, causing the cells to multiply faster which, in turn, causes a faster cosmetic result.

An ideal cosmetic and/or a pharamceutical preparation aimed at treating aging skin and wrinkles should include PDGF, IGFs, and EGF. PDGF targets fibroblasts and cells of mesodermal orgin². The IGFs target fibroblasts and epithelia². EGF targets fibroblasts and epithelia². The combination of PDGF, IGFS-I, IGF-II, and EGF can cooperate collectively in overcoming the G1 restriction points in the cell cycle, yielding a faster cosmetic result. A cosmetic and/or a pharmaceutical preparation containing all four growth factors would be significantly beneficial in the treatment of aging skin and wrinkles.

Method of Treating Aging Skin and Wrinkles with Growth Factors Derived from One's Own Blood:

This method of treating aging skin and wrinkles using one's own blood is defined as an autologous procedure. The rationale for using one's own blood to treat aging skin and wrinkles is because it is rich in a combination of growth factors. The plasma and serum are rich in growth factors that target fibroblasts and epithelia. The growth factors, PDGF, and IGFs I and II are found in the blood^{2,4,5}. PDGF targets fibroblasts and cells of mesodermal orgin². The IGFs target fibroblasts and epithelia². The alpha granules of platelets contain PDGF^{2,3,4}. The PDGF is released from platelets when blood clots^{2,5}. PDGF is in high concentrations in the serum⁵. Serum is obtained from whole blood when it is allowed to stand in a tube that does not contain an anticoagulant. The plasma is rich in IGFs. It was noted earlier that the plasma levels of IGF-I declines with age 5. Whereas, IGF-I plasma levels remain high throughout one's life⁵. One would expect the plasma levels of IGF-I to be lower for older individuals, especially those over the age of 30⁵. It is interesting that at about age 30 the tell-tale of age begins in the face, especially in women. This is not a coincidence, the GH and IGF-I blood levels begin to decline in a parallel fashion and significantly at age 30⁵. It appears from the literature that a decline in blood levels of IGF-I may be one cause why skin ages.

Ideally, if a cosmetic preparation aimed at treating aging skin and wrinkles using one's own blood, it should be obtained from the individual before he or she reaches age 30. The younger the individual, the higher their IGF-I blood levels. The blood contains both PDGF, IGF I, and IGF II^{2,3,4,5}. The three growth factors can cooperate collectively to overcome the G1 restriction points in the cell cycle, and yield a faster cosmetic result. Method of collecting blood and preparation of the serum and plasma: Whole blood is collected from an individual via venipuncture by a phlebotomist. After the blood is drawn, it is divided equally and placed in separate glass tubes. One tube consist of an anticoagulant, and the other does not. The plasma is collected from the whole blood in the tube containing the anticoagulant after it is centrifuged. The plasma is transferred from the tube with a pipette and placed into another glass tube. The serum is collected from the whole blood after it clots. The clotted blood is centrifuged and the serum from it is transferred into the tube containing the plasma and, both are mixed thoroughly. The tube containing the mixture of plasma and serum is refrigerated until further use. The mixture contains PDGFand IGFs. The mixture containing the growth factors can be incorporated into a topical face cream or lotion.

Mode of Delivery of Growth Factors To the Deep Layers of the Skin:

The mixture can also be incorporated into the aqueous chambers of liposomes commercially prepared from phosphatidylcholine (lecithin). Liposomes are used widely in the cosmetic industry because the manufacturers claim that they can penetrate deeply into the skin. The development of multivesicular lipid-based carrier liposome technology provides for sustained and controlled delivery of therapeutic agents^{8,9,10,11}. It also permits high drug loading and modulation of drug release rates^{8,9,10,11}. Liposomes appears to be excellent vehicles for the delivery of growth factors.into the deep layers of the skin. Thus, assuring that the growth factors reach their target cells in the deep layers of the skin. The liposomal mode of delivery of the growth factors, IGF-I and IGF-II may be the best vehicular method to assure their delivery to target cells. It was noted earlier that IGFs circulate in complex with carrier proteins which facilitate their delivery to the receptors on target cells. In order for a topical cosmetic and/or a pharmeceutical preparation utilizing growth factors to effectively treat aging skin and wrinkles, it must provide for an effective mode of delivery in order for the growth factors to reach their receptors.

Method of Treating Aging Skin and Wrinles Using a Combination of Commercially Prepared Growth Factors:

PDGF, EGF and IGFs can be commerically prepared using the technology of recombinant DNA. These growth factors can be obtained in high yield and high purity using the technology of recombinant DNA. These commercially prepared growth factors then can be incorporated into topical cosmetic and/or pharmaceutical preparations and applied to the face for the treatment of aging skin and wrinkles. The commerically prepared growth factors PDGF,EGF and IGF-I and IGF-II could be incorporated into a solution and injected directly into the skin with a fine needle to treat aging skin and wrinkles. Because the growth factors are are relatively small polypeptides and are soluble in water, they could be readily incorporated into the aqueous chambers of liposomes and delivered to the deep layers of the skin.

### References

1. Marks Dawn B, Ph.D., Marks, Allan D., M. D. and Smith, Colleen M., Ph.D.: Basic Medical Biochemistry. Lipincott Williams and Wilkins, Philadelphia, 1966, P. 172, 179, 228, 252, 262-263, 131, 683, 735-736, 738.

2. Davis, Bernard D., M. D., et al.: Microbiology. 4th Edition. J, B. Lipincott Company, Philadelphia, 1990, P. 836-839.

3 Schwartz, Seymour I., M. D., Shires, Tom G., M. D. and Spencer Frank C., M. D.: Principles of Surgery. Fifth Edition. McGraw-Hill, Inc., 1989, P. 35, 313- 318, 337.

4. Wyngaarden, M. D. and Smith, Lloyd H. Jr., M. D.: Cecil Textbook of Medicine. W. B. Saunders Company, Philadelphia, 1988, P.158-161, 322-323.

5. Wilson, Jean D. M. D. et. al.: Williams Textbook of Endocrinology. 9th Edition. W. B. Saunders Company, Philadelphia, 1998, P. 101-102, 118-119, 122,267, 1288-1289.

6. Lehninger, Albert L., Nelson, David L., and Cox, Michael M. Principles of Biochemistry. 2nd. Edition. Worth Publishers, Inc. New York, 1993, P. 135, 171-174, 235, 314.

7. Stryer, Lubert.: Biochemistry. 3rd. Edition. W. H. Freeman and Company, New York, P. 264-271, 274-277.

8. Kim. S., et al., “Preparation of Multivesicular Liposomes”. Biochem. Biophys. Acta. 728, 339-348 (1983).

9. Lasic, D. D., “liposomes: Synthetic Lipid Microsperes Serve as Multipurpose Vehicles for the Delivery of Drugs, Genetic Material and Cosmetics, “Am. Sci. 80, 20-31 (1992).

10. Katre, N. V. et al., “A Multivesicular Lipid-Based, Sustained-Rlease System for the Delivery of Therapeutic Proteins, “Pharmacol. Eng. pp. 8-18 (March-April 1999).

11. Ye, Q., Katre, N. and Sankaram, “Modulation of Drug Loading in Multivesicular Liposomes,” U.S. Pat. No. 6,106,858 (2000). Hill,1997, P.119-120.

What is claimed is:

1. A method of treating aging skin and wrinkles in a human with a combination of commercially prepared growth factors, the method comprising the step of combining platelet derived growth factor (PDGF), epidermal growth factor (EGF), and insulin-like growth factors, IGF-I, and IGF-II in effective therapeutic concentrations and incorporating them into a suitable cosmetic and/or a pharmaceutical preparation, and applying said preparation to said human's face to stimulate regeneration of skin cells in the basal layer of the epidermis and their migration to the surface, to induce resting fibroblasts in the dermis to divide and synthesize elastin, collagen, proteoglycans, and enhance the production of new ground substance, thereby reducing wrinkles and enhancing the clarity, resiliency, elasticity, and suppleness of said human's skin.

2. A method of treating aging skin and wrinkles in a human by using said human's own blood, having in it a combination of platelet derived growth factor (PDGF), IGF-I and IGF-II, the method comprising the step of collecting said human's whole blood by venipuncture and preparing from it serum and plasma, then combining the plasma and serum into a single mixture, and incorporating said mixture into a suitable cosmetic and/or a pharmaceutical preparation, and applied to the face of said human to stimulate regeneration of skin cells in the basal layer of the epidermis and their migration to the surface, to induce resting fibroblastic cells in the dermis to divide, multiply, and synthesize elastin, collagen, proteoglycans, and enhance the production of new ground substance, thereby reducing wrinkles, enhancing the clarity, elasticity, resiliency, and suppleness of said human's skin.
